(19) United States
(12) Patent Application Publication
(10) Pub. No.: US 2025/0009348 A1
(43) Pub. Date: Jan. 9, 2025

(54) HUB LOCK FOR A TISSUE PERFORATION DEVICE

(71) Applicant: Boston Scientific Medical Device Limited, Galway (IE)

(72) Inventors: Brock Miller, Toronto (CA); Kai-Lon Fok, Mississauga (CA); Matthew Gravett, London (CA)

(21) Appl. No.: 18/763,062

(22) Filed: Jul. 3, 2024

Related U.S. Application Data
(60) Provisional application No. 63/512,409, filed on Jul. 7, 2023.

(51) Int. Cl. A61B 17/00; A61M 25/09

(52) U.S. Cl. CPC A61B 17/0057; A61B 2017/00247; A61M 2025/09125

(57) ABSTRACT

A perforation system for accessing a location in or near a patient's heart is disclosed. The system includes a puncture device having a proximal portion and a distal portion including a distal tip. The system further includes an elongated introducer defining a lumen adapted to receive and allow longitudinal translation of the puncture device therein, the elongated introducer having an introducer proximal portion and an introducer distal portion, the introducer proximal portion including a locking hub. The locking hub includes a slot and an engaging surface configured so that the proximal portion can be positioned in the slot and against the engaging surface, so as to generate a securing frictional force between the proximal portion and the engaging surface, the securing friction force sufficient to inhibit the longitudinal translation of the puncture device with respect to the elongated introducer.

### CROSS REFERENCE TO RELATED APPLICATION

The present application claims priority to U.S. Provisional Patent Application No. 63/512,409, filed Jul. 7, 2023, the entire disclosure of which is incorporated herein by reference.

### TECHNICAL FIELD

The present invention relates generally to methods and devices for accessing anatomical structures in the body of a patient. More specifically, the present invention is concerned with a hub lock for use in combination with a tissue perforation system.

### BACKGROUND

Devices currently exist for creating a puncture, channel, or perforation within a tissue located in a body of a patient. A radiofrequency (RF) guidewire used for accessing a location in or near the patient's heart, for example epicardial or transseptal access, involves the RF guidewire being inserted in an opening at the proximal end of an introducer and guided through the introducer until the guidewire emerges from the distal end of the introducer with a RF electrode on the tip of the RF guidewire protruding from the distal end of the introducer. In this case, the RF guidewire is free to move forward and backwards into the introducer. The precise positioning of the electrode tip of the RF guidewire is required for accurate and repeatable puncture of a target site.

Some currently available devices include a collar that is placed behind the tip electrode of the RF guidewire which is used as a hard stop to position the electrode at the precise position relative to the rigid sheath. Thus, once a user is ready to advance the RF guidewire using the rigid sheath, the force of advancing the rigid sheath will be transmitted to the RF guidewire through the electrode collar so that the proper electrode positioning on the target site is maintained relative to the rigid sheath while a force is applied to advance the rigid sheath into a patient. Other current devices are positioned using a proximal marker. Such devices, however, do not have a mechanism to ensure the electrode tip remains in place at the tip of the introducer when there is a force acting on the tip electrode in the direction away from the introducer sheath. This force can include the electrode being pinched or caught on tissue while the introducer is being retracted or the user unintentionally pushing the RF guidewire forward. Additionally, the tip electrode may be pushed back inside the introducer and once inside the introducer and/or the dilator the tip is no longer in contact with tissue and results in failure to puncture or perforate a tissue.

Against this background, there exists a continuing need in the industry to provide improved locking mechanism for radiofrequency perforation devices and methods. An object of the present invention is therefore to provide such a method and device.

### SUMMARY

In Example 1, a perforation system for accessing a location in or near a patient's heart includes a puncture device having a puncture device proximal portion and a puncture device distal portion including a distal tip; and an elongated introducer defining a lumen adapted to receive and allow longitudinal translation of the puncture device therein, the elongated introducer having an introducer proximal portion and an introducer distal portion, the introducer proximal portion including a locking hub; wherein the locking hub includes a slot and an engaging surface configured so that the puncture device proximal portion can be positioned in the slot and against the engaging surface, so as to generate a securing frictional force between the puncture device proximal portion and the engaging surface, the securing friction force sufficient to inhibit the longitudinal translation of the puncture device with respect to the elongated introducer.

Example 2 is the perforation system of Example 1, wherein the slot is a J-shaped opening.

Example 3 is the perforation system of any of Examples 1-2, wherein the slot further includes an entry slot.

Example 4 is the perforation system of any of Examples 1-3, wherein the slot further includes a locking slot.

Example 5 is the perforation system of any of Examples 1-4, wherein the puncture device proximal portion can be positioned in the entry slot and bent towards the locking slot.

Example 6 is the perforation system of any of Examples 1-5, wherein the puncture device proximal portion bends at about 45 degrees to 90 degrees with respect to the locking hub.

Example 7 is the perforation system of any of Examples 1-5, wherein the puncture device is an RF puncture wire having an electrically active distal tip and the proximal portion of the RF puncture wire is inserted into the slot via the entry slot and is manipulated by a user into the locking slot for the RF puncture wire to be locked therein.

Example 8 is the perforation system of Example 1, wherein the elongated introducer may include multiple slots on the locking hub.

Example 9 is the perforation system of Example 1, wherein the puncture device is a Nitinol radiofrequency guidewire.

Example 10 is the perforation system of Example 1, wherein the elongated introducer further includes a dilator.

Example 11 is the perforation system of Example 1, wherein the engaging surface promotes friction with the puncture device.

Example 12 is the perforation system of Example 1, wherein the engaging surface is an interior surface of the slot that engages the proximal portion of the puncture device.

Example 13 is the perforation system of Example 1, wherein the engaging surface is the interior surface of the locking slot that secures with the puncture device.

Example 14 is the perforation system of Example 1, wherein the engaging surface is made of a high coefficient of friction material to enhance the frictional force between the engaging surface and the puncture device.

Example 15 is the perforation system of Example 1, wherein the engaging surface includes a surface finish that promotes friction with the puncture device.

In Example 16, a radiofrequency perforation system for accessing a location in or near a patient's heart includes a radiofrequency puncture wire having a wire proximal portion and a wire distal portion including an electrically active distal tip. The radiofrequency perforation system also includes an elongated introducer defining a lumen adapted to receive and allow longitudinal translation of the radiofrequency puncture wire therein, the elongated introducer having an introducer proximal portion and an introducer distal portion, the introducer proximal portion including a locking hub; wherein the locking hub includes a slot and an engaging surface configured so that the wire proximal portion can be positioned in the slot and against the engaging surface, so as to generate a securing frictional force between the wire proximal portion and the engaging surface, the securing friction force sufficient to inhibit the longitudinal translation of the radiofrequency puncture wire with respect to the elongated introducer.

Example 17 is the radiofrequency perforation system of Example 16 wherein the slot is a J-shaped opening.

Example 18 is the radiofrequency perforation system of Example 17 wherein the slot further includes an entry slot.

Example 19 is the radiofrequency perforation system of Example 18 wherein the slot further includes a locking slot.

Example 20 is the radiofrequency perforation system of Example 19 wherein the wire proximal portion can be positioned in the entry slot and bent towards the locking slot.

Example 21 is the radiofrequency perforation system of Example 20 wherein the wire proximal portion bends at about 45 degrees to 90 degrees with respect to the locking hub.

Example 22 is the radiofrequency perforation system of Example 16 wherein the elongated introducer may include multiple slots on the locking hub.

Example 23 is the radiofrequency perforation system of Example 16 wherein the radiofrequency puncture wire is a Nitinol radiofrequency guidewire.

Example 24 is the radiofrequency perforation system of Example 16 wherein the engaging surface is an interior surface of the slot that engages the wire proximal portion of the RF puncture wire.

Example 25 is the radiofrequency perforation system of Example 16 wherein the engaging surface is made of a high coefficient of friction material to enhance the frictional force between the engaging surface and the RF puncture wire.

In Example 26, a perforation system for epicardial or transseptal access includes a dilator having a dilator body defining a dilator lumen and a tapered distal tip; a puncture device having a proximal portion and a distal portion including a distal tip; and an elongated introducer defining a lumen adapted to receive and allow longitudinal translation of the puncture device therein, the elongated introducer having an introducer proximal portion and an introducer distal portion, the introducer proximal portion including a locking hub; wherein the locking hub includes a slot and an engaging surface configured so that the proximal portion can be positioned in the slot and against the engaging surface, so as to generate a securing frictional force between the proximal portion and the engaging surface, the securing friction force sufficient to inhibit the longitudinal translation of the puncture device with respect to the elongated introducer.

Example 27 is the perforation system of Example 26, wherein the slot is a J-shaped opening.

Example 28 is the perforation system of Example 27, wherein the slot further includes an entry slot and a locking slot.

Example 29 is the perforation system of Example 28, wherein the proximal portion of the puncture device is inserted into the slot via the entry slot and is manipulated by a user into the locking slot for the puncture device to be locked therein.

Example 30 is the perforation system of Example 29, wherein the proximal portion bends at about 45 degrees to 90 degrees with respect to the locking hub.

Example 31 is the perforation system of Example 26, wherein the elongated introducer may include multiple slots on the locking hub.

Example 32 is the perforation system of Example 26, wherein the puncture device is a radiofrequency puncture wire.

Example 33 is the perforation system of Example 26, wherein the engaging surface is an interior surface of the slot that engages the proximal portion of the puncture device.

Example 34 is the perforation system of Example 26, wherein the engaging surface is made of a high coefficient of friction material to enhance the frictional force between the engaging surface and the puncture device.

In Example 35, a method of making a radiofrequency perforation system for accessing a location in or near a patient's heart includes providing a radiofrequency puncture wire having a wire proximal portion and a wire distal portion including an electrically active distal tip. The method of making a radiofrequency perforation system also includes advancing an elongated introducer defining a lumen adapted to receive and allow longitudinal translation of the radiofrequency puncture wire therein, the elongated introducer having an introducer proximal portion and an introducer distal portion, the introducer proximal portion including a locking hub; wherein the locking hub includes a slot and an engaging surface configured so that the wire proximal portion can be positioned in the slot and against the engaging surface, so as to generate a securing frictional force between the wire proximal portion and the engaging surface, the securing friction force sufficient to inhibit the longitudinal translation of the radiofrequency puncture wire with respect to the elongated introducer.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A and 1B are schematic illustrations of a medical procedure within a patient's heart for gaining access to the transseptal and epicardial space, respectively, according to embodiments of the present disclosure.

FIG. 2 is a perspective view of a radiofrequency puncture wire inserted into an introducer having a locking hub, according to embodiments of the present disclosure.

FIGS. 3A-3E are partial perspective and enlarged views of the locking hub and the radiofrequency puncture wire of FIG. 2, according to embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

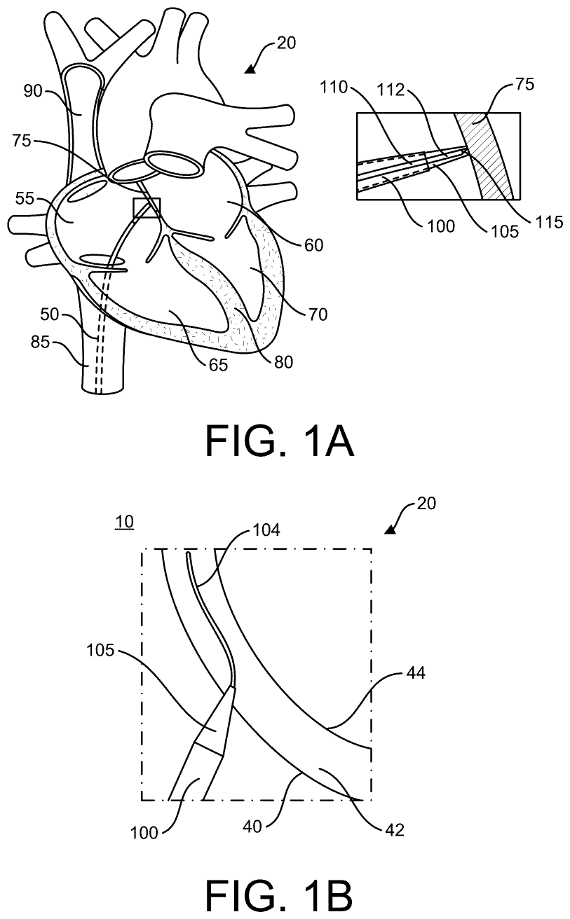

FIGS. 1A and 1B are schematic illustrations of a medical procedure within a patient's heart for gaining access to the transseptal and epicardial space, according to embodiments of the present disclosure. FIG. 1A is an illustration of a medical procedure 10 within a patient's heart 20 utilizing a transseptal access system 50. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

Procedures for providing access to the left atrium 60 use transseptal access systems and devices for subsequent deployment of the aforementioned diagnostic and/or therapeutic devices within the left atrium 60. In these procedures, a target tissue site can be defined by tissue on the atrial septum 75. The target site is accessed via the inferior vena cava (IVC) 85, for example through the femoral vein, according to conventional catheterization techniques. In other embodiments, access to the target site on the atrial septum 75 may be accomplished using a superior approach wherein the transseptal access system 50 is advanced into the right atrium 55 via the superior vena cava (SVC) 90.

Transseptal access system procedures may include many devices like an introducer sheath 100, a dilator 105, a puncture device having distal end portion 112 terminating in a tip electrode 115, and a guidewire. In various embodiments, the puncture device 110 is a mechanical puncture device (e.g., a needle) or an RF perforation device. The puncture device 110 can be disposed within the dilator 105, which itself can be disposed within the sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator may then be introduced into the sheath and over the guidewire, and advanced through the sheath into the SVC. Alternatively, the dilator may be fully inserted into the sheath prior to entering the body, and both may be advanced simultaneously towards the heart.

When the guidewire, sheath 100 and dilator 105 have been positioned in the SVC 90, the guidewire is removed from the body, and the sheath 100 and the dilator 105 are retracted so that their distal ends are positioned in the right atrium 55. The puncture device 110 described can then be introduced into the dilator 105, and advanced toward the heart 20. The puncture device 110 is then positioned such that the tip electrode 115 is aligned with or protruding slightly from the distal end of the dilator 105. In embodiments where the puncture device 110 is an RF perforation device, with the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 75 V (peak-to-peak), and functions to vaporize cells in the vicinity of the tip electrode, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 second and about 5 seconds.

Still another medical procedure 10 developed for diagnosing or treating physiological ailments originating within a heart 20 includes epicardial ablation to help restore a regular heart rhythm, as shown in FIG. 1B. As illustrated, the heart 20 includes a pericardium 40, a pericardial cavity 42 and a myocardium 44. The heart 20 is typically approached using a subxiphoid approach. Epicardial access is achieved via puncturing a layer of the pericardium 40 while avoiding the myocardium 44 of the heart. The pericardium 40 is a tough, double-walled, fibroelastic sac encompassing the heart 20 and the roots of the great vessels. The pericardium 40 includes two layers, an outer layer made of strong connective tissue often referred to as the fibrous pericardium, and an inner layer made of serous membrane often referred to as the serous pericardium. The mesothelium, or mesothelial cells, that constitutes the serous pericardium also covers the myocardium of the heart as epicardium, resulting in a continuous serous membrane invaginated onto itself as two opposing surfaces such as over the fibrous pericardium 40 and over the heart 20. This creates a pouch-like virtual or potential space around the heart 20 enclosed between the two opposing serosal surfaces, often referred to as the pericardial space or pericardial cavity 42.

In some embodiments, the pericardium 40 may be punctured with a puncture device 110, such as a needle (or other mechanical puncture device). Once punctured, a dilator 105 is advanced to dilate the puncture created by the needle through the pericardium 40. In certain embodiments, a sheath 100 may be advanced with the dilator 105 simultaneously. In other embodiments, the sheath 100 may be advanced afterwards. The sheath 100 and the dilator 105 may then be withdrawn to leave the guidewire 104 in the pericardial cavity 42. Minimally invasive access to the epicardium is required for diagnosis and treatment of a variety of arrhythmias and other conditions. During epicardial ablation, tiny scars are created on the outside of the heart to create a transmural lesion. In other words, to achieve an ablated tissue through the thick muscle of the heart.

The present disclosure describes novel devices and methods for providing safe access to the heart, specifically transseptal and epicardial access, using radiofrequency energy. As will be explained in greater detail herein, the embodiments of the present disclosure simplify the means of puncturing the heart while providing a mechanism to ensure that the tip electrode of the guidewire remains in place and also providing enhanced manipulability by the user.

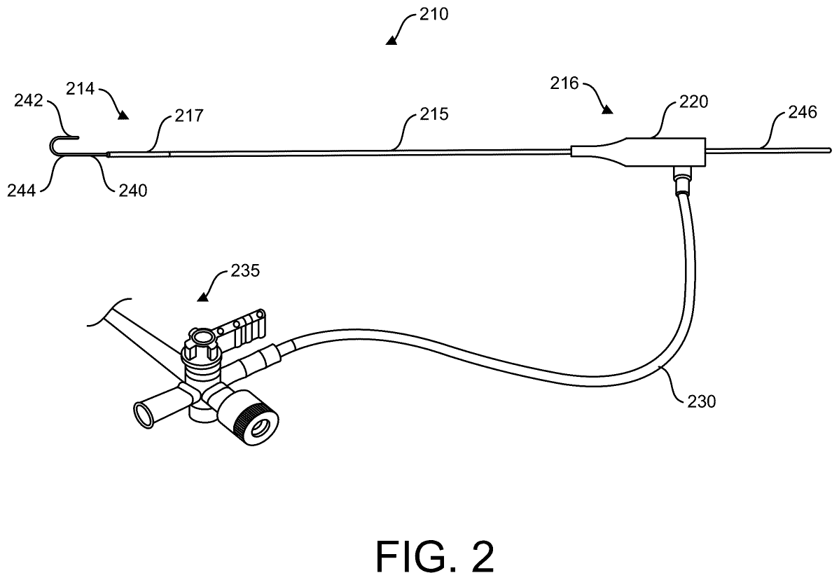

FIG. 2 is a perspective view of a puncture device 240 inserted into an elongated introducer 210 for accessing targeted regions in a patient's heart, according to embodiments of the present disclosure. While described below as an RF puncture wire, in various embodiments, the puncture device 240 is a mechanical puncture device (e.g., a needle). As shown, the RF puncture wire 240 includes a wire proximal portion 246 and a wire distal portion 214 terminating in an electrically active distal tip electrode 242. Additionally, as shown, the elongated introducer 210 defines a lumen and includes an introducer proximal portion 216 and an introducer distal portion 214. The introducer lumen (not shown) extends longitudinally through the introducer 210 from the introducer proximal portion 216 to the introducer distal portion 214. The introducer 210 serves to receive and allow a longitudinal translation of the RF puncture wire 240 therein. As shown, a locking hub 220 is connected to the most proximal end of the introducer proximal portion 216. In some embodiments, the locking hub 220 includes a tapered distal portion and may be configured to couple to ancillary devices. In shown embodiments, a rigid sheath 215 defining a lumen is connected to the distal portion of the locking hub 220. In some embodiments and as shown in FIG. 2, a dilator 217 may be connected to the distal end of the sheath 215. As shown in FIG. 2, the RF puncture wire 240 may be inserted into the introducer 210 at the introducer proximal portion 216 through the locking hub 220 and exit through the introducer distal portion 214.

In some embodiments, the introducer 210 may also include a side tube 230 that is connected to a stop cock delivery valve 235. The introducer 210 stop cock 235 may connect to at least one entry line of the introducer 210 and can further connect to a fluid source thereby providing a manipulable delivery interface from the fluid source to the introducer 210. In an embodiment, contrast agents/media may be delivered into the space to confirm access of the RF puncture wire 240. In an embodiment, how the contrast agent is depicted under fluoroscopy may inform a user the location of the RF puncture wire 240. In an embodiment, the contract may include, but is not limited to, iodine-based contrast agents, gadolinium-based contrast agents, microbubble contrast agents, iron oxide nanoparticles, among others. In some embodiments, the sheath 215 may preferably be a tubular member having a substantially uniform outer shape at the proximal end of the sheath 215. In certain embodiments, the sheath 215 may further have a distal taper tapering at the introducer distal portion 214. In some embodiments, the dilator 217 may further include a distal taper tapering at the introducer distal portion 214. In some embodiments, the introducer 210 may be used without the dilator 217.

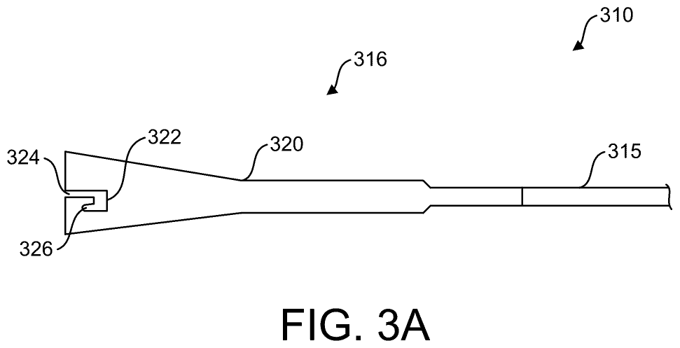

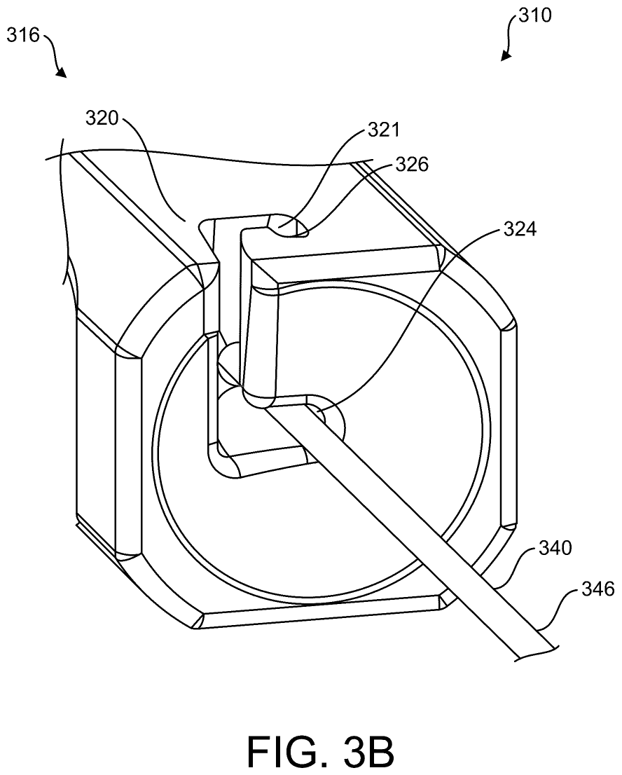

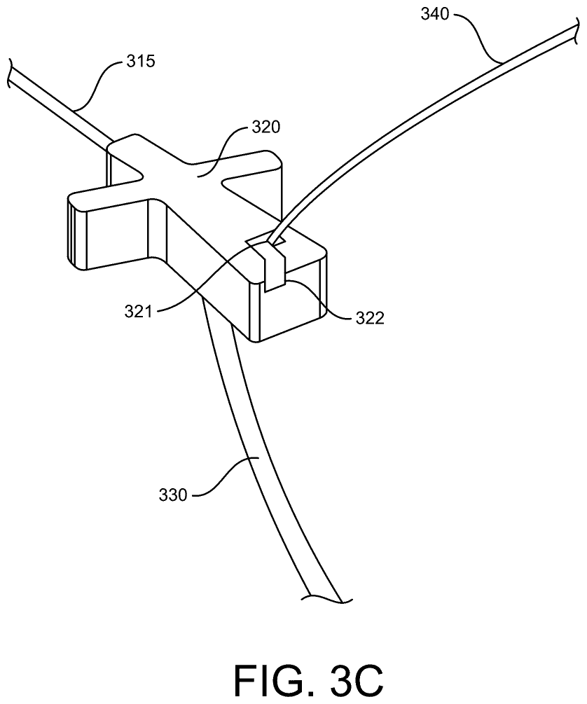

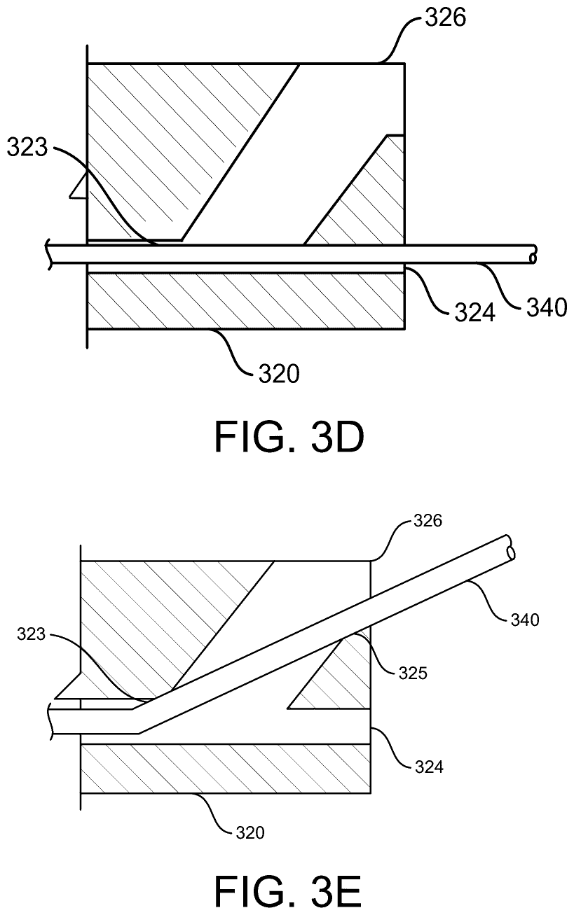

FIGS. 3A-3E are partial perspective and enlarged views of an introducer 310 having a proximal portion 316, according to embodiments of the present disclosure. The introducer 310 of FIGS. 3A-3E is functionally and structurally similar to the introducer 210 of FIG. 2. The introducer 310 defines a lumen (not shown) and includes a locking hub 320 having a distal taper that connects to a sheath 315. The introducer lumen extends longitudinally through the introducer 310. As shown in FIG. 3B, the introducer 310, and hence the locking hub 320, serve to receive and allow a longitudinal translation of a RF puncture wire 340 therein. As shown in FIGS. 3B and 3C, in certain embodiments, the locking hub 320 operates to maintain a proximal portion 346 of a RF puncture wire 340 in place. The locking hub 320 includes an engaging surface 321 and a J-shaped slot 322 having an entry slot 324 and a locking slot 326. In certain embodiments, as shown in FIGS. 3B and 3C, the engaging surface 321 includes the interior surface of the slot 322 and is the entire area within the locking hub 320 that engages with the proximal portion 346 of the RF puncture wire 340 once it is inserted into the locking hub 320. In other embodiments, the engaging surface 321 is the interior surface of the locking slot 326, which is the upper portion of the slot 322, that secures the RF puncture wire 340.

As shown in FIG. 3B, during use, the proximal portion 346 of the RF puncture wire 340 is inserted into the J-shape slot 322 via the entry slot 324 and is then manipulated by a user by sliding the RF puncture wire 340 into the locking slot 326 for the puncture wire to be locked therein. In some embodiments, the slot 322 and the engaging surface 321 of the locking hub 320 are configured so that the RF puncture wire proximal portion 324 may be positioned in the slot 322 and against the engaging surface so as to generate a securing frictional force between the wire proximal portion 346 and the engaging surface 321. In some embodiments, the securing friction force is sufficient to inhibit the longitudinal translation of the RF puncture wire 340 with respect to the elongated introducer 310. As shown in FIG. 3D, when the RF puncture wire 340 is inserted into the locking hub 320, the RF puncture wire will have a first, initial unlocked position. As described above and shown in FIG. 3E, when the RF puncture wire 340 is manipulated into the locking slot 326 it will assume a second, locked position. Thus, in some embodiments, the points of contact between the RF puncture wire 340 and the locking hub 320 will be contact point 323, at the base of the J-shaped slot 322, and contact point 325, at upper surface of the locking slot 326 when the RF puncture wire is in the locked position.

In certain embodiments, the slot 322 allows the distal portion of the RF puncture wire 340, and hence the active tip electrode, to lock in place relative to the introducer in order to ensure accurate and repeatable puncture to a target tissue as desired. Thus, a force acting on the RF puncture wire 340 in the opposite direction of the introducer would be counteracted by the locking mechanism of the locking hub 320, allowing the tip electrode of the RF puncture wire 340 to maintain its ideal position relative to the introducer 310. In some embodiments, the RF puncture wire 340 includes an adequate combination of stiffness, hook-lock angle, and sufficient distance between the slot and where the wire exits the introducer. In some embodiments, the engaging surface 321 is made of a high coefficient of friction material to enhance the frictional force between the engaging surface 321 and the RF puncture wire 340. In an embodiment, the enhanced frictional force may be placed on the introducer hub. In other embodiments, the enhanced frictional force may be placed on certain locations along the puncture wire 340. In these embodiments, the engaging surface 321 may be made of polyurethane, which is known for its friction characteristics, acrylonitrile butadiene styrene (ABS), which is a thermoplastic known for its strength and durability, polycarbonate, which is a transparent, impact-resistant plastic that can be modified to enhance its friction properties, polypropylene, which is a versatile and cost-effective plastic used in a wide range of medical applications, among others.

Similarly, in some embodiments, the engaging surface 321 includes a surface finish (e.g., a texture) that promotes friction with the RF puncture wire. In these embodiments, the engaging surface 321 may have a textured or rough surface. Adding texture or creating a rough surface on plastic can increase the friction. This can be achieved through methods like embossing, etching, or using abrasive materials during the molding process. The texture can range from fine to coarse, depending on the required level of friction. In other embodiments, the engaging surface 321 may include a micro-structed surface, which involves creating tiny patterns or features on the plastic surface. These microstructures, such as grooves or ridges, can increase the contact area between the surface and the object in contact, resulting in improved friction. In still other embodiments, the engaging surface 321 may have a matte or stain finish, which can provide a certain level of friction. These finishes have a slightly rough texture compared to glossy finishes, which can enhance grip and friction. In still other embodiments, the engaging surface 321 may include a surface treatment such as corona treatment or plasma treatment that can alter the surface properties of plastics. These treatments can increase surface energy and improve the adhesion of other materials, which may indirectly affect friction by influencing the interaction between the surface and the contacting objects. In various embodiments, the engaging surface may be located on a portion of the RF wire in addition to or as an alternative to the engaging surface located on the introducer hub.

In some embodiments, once the RF puncture wire 340 hooks-and-locks (see FIG. 3E), the wire bends at an angle of between about 10 degrees to 60 degrees with respect to the locking hub 320. In certain embodiments, the RF wire 340 bends at about 20 degrees to 50 degrees with respect to the locking hub 320. In some embodiments, the RF puncture wire 340 may be made of a Nitinol RF guidewire to allow for the needed flexibility to bend and stiffness to remain in place in the locking hub 320. In further embodiments, the introducer 310 may include multiple slots 322 on the locking hub 320 to ensure further security of the RF puncture wire 340.

In an embodiment, the puncture wire 340 may include line markers placed in specific locations on the puncture wire 340. In some embodiments, the line markers on the puncture wire 340 may indicate the location of the puncture wire 340 that is locked. In an embodiment, the line marker may be made of heat shrink material. In some embodiments, each line marker may be a specific distance away from the other so that a user may precisely indicate the location in which the puncture wire 340 is locked and the amount of the puncture wire 340 that is inserted.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A radiofrequency perforation system for accessing a location in or near a patient's heart, the system comprising:
a radiofrequency puncture wire having a wire proximal portion and a wire distal portion including an electrically active distal tip; and
an elongated introducer defining a lumen adapted to receive and allow longitudinal translation of the radiofrequency puncture wire therein, the elongated introducer having an introducer proximal portion and an introducer distal portion, the introducer proximal portion including a locking hub;
wherein the locking hub includes a slot and an engaging surface configured so that the wire proximal portion can be positioned in the slot and against the engaging surface, so as to generate a securing frictional force between the wire proximal portion and the engaging surface, the securing friction force sufficient to inhibit the longitudinal translation of the radiofrequency puncture wire with respect to the elongated introducer.

2. The radiofrequency perforation system of claim 1, wherein the slot is a J-shaped opening.

3. The radiofrequency perforation system of claim 2, wherein the slot further includes an entry slot.

4. The radiofrequency perforation system of claim 3, wherein the slot further includes a locking slot.

5. The radiofrequency perforation system of claim 4, wherein the wire proximal portion can be positioned in the entry slot and bent towards the locking slot.

6. The radiofrequency perforation system of claim 5, wherein the wire proximal portion bends at about 45 degrees to 90 degrees with respect to the locking hub.

7. The radiofrequency perforation system of claim 1, wherein the elongated introducer may include multiple slots on the locking hub.

8. The radiofrequency perforation system of claim 1, wherein the radiofrequency puncture wire is a Nitinol radiofrequency guidewire.

9. The radiofrequency perforation system of claim 1, wherein the engaging surface is an interior surface of the slot that engages the wire proximal portion of the RF puncture wire.

10. The radiofrequency perforation system of claim 1, wherein the engaging surface is made of a high coefficient of friction material to enhance the frictional force between the engaging surface and the RF puncture wire.

11. A perforation system for epicardial or transseptal access, the system comprising:
a dilator having a dilator body defining a dilator lumen and a tapered distal tip;
a puncture device having a proximal portion and a distal portion including a distal tip; and
an elongated introducer defining a lumen adapted to receive and allow longitudinal translation of the puncture device therein, the elongated introducer having an introducer proximal portion and an introducer distal portion, the introducer proximal portion including a locking hub;
wherein the locking hub includes a slot and an engaging surface configured so that the proximal portion can be positioned in the slot and against the engaging surface, so as to generate a securing frictional force between the proximal portion and the engaging surface, the securing friction force sufficient to inhibit the longitudinal translation of the puncture device with respect to the elongated introducer.

12. The perforation system of claim 11, wherein the slot is a J-shaped opening.

13. The perforation system of claim 12, wherein the slot further includes an entry slot and a locking slot.

14. The perforation system of claim 13, wherein the proximal portion of the puncture device is inserted into the slot via the entry slot and is manipulated by a user into the locking slot for the puncture device to be locked therein.

15. The perforation system of claim 14, wherein the proximal portion bends at about 45 degrees to 90 degrees with respect to the locking hub.

16. The perforation system of claim 11, wherein the elongated introducer may include multiple slots on the locking hub.

17. The perforation system of claim 11, wherein the puncture device is a radiofrequency puncture wire.

18. The perforation system of claim 11, wherein the engaging surface is an interior surface of the slot that engages the proximal portion of the puncture device.

19. The perforation system of claim 11, wherein the engaging surface is made of a high coefficient of friction material to enhance the frictional force between the engaging surface and the puncture device.

20. A method of making a radiofrequency perforation system for accessing a location in or near a patient's heart, the method comprising:
providing a radiofrequency puncture wire having a wire proximal portion and a wire distal portion including an electrically active distal tip; and
advancing an elongated introducer defining a lumen adapted to receive and allow longitudinal translation of the radiofrequency puncture wire therein, the elongated introducer having an introducer proximal portion and an introducer distal portion, the introducer proximal portion including a locking hub;
wherein the locking hub includes a slot and an engaging surface configured so that the wire proximal portion can be positioned in the slot and against the engaging surface, so as to generate a securing frictional force between the wire proximal portion and the engaging surface, the securing friction force sufficient to inhibit the longitudinal translation of the radiofrequency puncture wire with respect to the elongated introducer.
